(19) United States
(12) Patent Application Publication
(10) Pub. No.: US 2024/0277368 A1
(43) Pub. Date: Aug. 22, 2024

(54) SURGICAL EQUIPMENT

(71) Applicant: KYOWA INDUSTRIAL CO., LTD., Takasaki-shi, Gunma (JP)

(72) Inventors: Haruo TSUJI, Takasaki-shi, Gunma (JP); Takuya KAWAWADA, Takasaki-shi, Gunma (JP); Toshiharu KIMURA, Takasaki-shi, Gunma (JP)

(21) Appl. No.: 18/570,261

(22) PCT Filed: Jun. 30, 2021
(86) PCT No.: PCT/JP2021/024747

§ 371 Date: Dec. 14, 2023

(51) Int. Cl. A61B 17/29

(52) U.S. Cl. CPC A61B 17/29; A61B 2017/2904; A61B 2017/2908

(57) ABSTRACT

Surgical equipment includes a pipe section to be inserted into an organism, a handle section extending in a direction nonparallel to a longitudinal direction of the pipe section, a transitioning section that couples a rear end portion of the pipe section and a front end portion of the handle section, and a treatment instrument disposed at a front end portion of the pipe section. The surgical equipment includes a manipulator section disposed in the handle section and manipulated when causing the treatment instrument to operate and a transmission member that connects the treatment instrument and the manipulator section and transmits a motion of the manipulator section to the treatment instrument. The transmission member includes a wire. The transitioning section has a hollow pipe shape through which the wire can be inserted. Only the wire is inserted through the transitioning section.

### TECHNICAL FIELD

The present invention relates to surgical equipment.

### BACKGROUND ART

In recent years, surgery with a reduced incision region has prevailed for a purpose of minimally invasion. In surgery in the neurosurgery field, for example, surgical equipment including a treatment instrument such as forceps is inserted into a lumen of a transparent tube (sheath) detained transcranially and, while checking of a diseased part such as a brain tumor being performed through the sheath with a microscope disposed right above the sheath, the treatment instrument is used to perform treatment.

As the surgical equipment used in the surgery explained above, for example, there is surgical equipment including a manipulator member gripped by a surgeon, an elastic member attached to one end of the manipulator member, front ends of the elastic member being crossed, and opening and closing members attached to the other end of the manipulator member and crossed in a shaft support section to be openable and closable, functional sections that perform incision, excision, grasping, holding, and cutting being included at front ends of the opening and closing members, the surgical equipment increasing and reducing a pressing force applied to the manipulator member to open and close the functional section (see, for example, Patent Literature 1). The surgical equipment of this type has an exterior shape formed by shifting an axis of the functional section at the front end and an axis of the manipulator member and coupling the functional section and the manipulator member via the opening and closing members. With such an exterior shape, it is intended to secure a visual field in performing surgery by a microscope and secure a space in a sheath.

The surgical equipment disclosed in Patent Literature 1 includes scissors as a treatment instrument. The scissors include a manipulator member that is capable of opening and closing and is manipulated when opening and closing the scissors.

As surgical equipment for performing precise surgery in surgery performed in a narrow surgical field via a sheath, for example, there is surgical forceps including a grasping section for a surgeon to insert fingers and a holding section that holds a diseased part (see, for example, Patent Literature 2). The surgical equipment of this type is composed of a bar-like forceps member and reduced in width and has an exterior shape formed by coupling the holding section and the grasping section at a front end via the forceps member and a crossing section.

As another example, there is micro forceps including a body section, a tubule, a core material, and a beak-like grasping section present at one front end of the core material, the micro forceps characterized in that fingers pushing in a push-in member are opened, whereby the tubule moves to an original position and the beak-like grasping section at the front end closes (see, for example, Patent Literature 3).

In the surgical equipment disclosed in Patent Literature 1, the manipulator member is coupled to a blade section via the opening and closing members and the shaft support section and has a structure in which the blade section moves in association with a movement of the manipulator member. The manipulator member is bent in an L shape to have a structure in which a visual field of a surgeon who observes a surgical field via a microscope is not hindered by a hand of the surgeon.

The surgical equipment disclosed in Patent Literature 2 includes scissors as a treatment instrument. The surgeon holds the grasping section with a same grasping method as a method of grasping general scissors and performs opening and closing of the holding section at the front end. The holding section has a structure in which the holding section is coupled to the grasping section via the forceps member and the crossing section and the holding section moves in association with a movement of the grasping section.

The surgical equipment disclosed in Patent Literature 3 includes a blade section as a treatment instrument for performing precise surgery. The blade section is coupled to the push-in member via the core material in the tubule, a fixing member, and a crank member. A surgeon holds the push-in member with a same grasping method as a method of grasping a pen and manipulates the push-in member to move the blade section that interlocks with the push-in member.

### CITATION LIST

### Patent Literature

Patent Literature 1: Japanese Patent No. 6013507
Patent Literature 2: Japanese Patent Application Laid-Open No. 2020-14489
Patent Literature 3: Japanese Patent Application Laid-Open No. 2018-175573

### SUMMARY OF INVENTION

### Technical Problem

However, the surgical equipment disclosed in Patent Literature 1 is large in instrument width and is unsuitable to be manipulated in a narrow sheath inserted for incision of a small region. In contrast, since the surgical equipment disclosed in Patent Literature 2 and Patent Literature 3 has bar-like structure, an occupied area in the sheath is minimized. However, in the surgical equipment disclosed in Patent Literature 2, since the surgeon grasps the grasping section with the same grasping method as the method of grasping scissors, precise manipulation is difficult in surgery requiring precision such as surgery in the neurosurgery field. It is difficult to perform precise surgery. In Patent Literature 3, precise manipulation can be performed because the surgeon grasps the push-in member with the same grasping method as the method of grasping a pen. However, since the blade section and the push-in member are present on the same axis in a longitudinal direction, a hand of the surgeon hinders the visual field at the time of a microscopic observation as explained above. Since several members are involved from driving of the push-in member before the blade section are moved, a precise movement of the push-in member by the surgeon is not appropriately transmitted to the blade section because of backlash of connection of the respective members.

An object of the present invention, which has been made in order to solve the problems with the related art, is to provide surgical equipment having an exterior shape formed by coupling a pipe section and a handle section via a transitioning section, the surgical equipment being capable of reducing hindrance of a visual field of a surgeon by the transitioning section and appropriately transmitting a precise movement of the handle section by the surgeon to a treatment instrument.

### Means for Solving Problem

Surgical equipment according to the present invention for achieving the object described above includes: a pipe section to be inserted into an organism; a handle section extending in a direction nonparallel to a longitudinal direction of the pipe section; a transitioning section that couples a rear end portion of the pipe section and a front end portion of the handle section; and a treatment instrument disposed at a front end portion of the pipe section. The surgical equipment includes: a manipulator section disposed in the handle section and manipulated when causing the treatment instrument to operate; and a transmission member that connects the treatment instrument and the manipulator section and transmits a motion of the manipulator section to the treatment instrument. The transmission member includes a wire. The transitioning section has a hollow pipe shape through which the wire can be inserted. Only the wire is inserted through the transitioning section.

### Advantageous Effects of the Invention

The surgical equipment of the present invention has an exterior shape formed by shifting an axis of the pipe section and an axis of the handle section and coupling the pipe section and the handle section via the transitioning section. The transitioning section has the hollow pipe shape and only the wire is inserted through the transitioning section. Therefore, the transitioning section can be reduced in size. It is possible to provide surgical equipment that reduces, through the reduction in the size of the transitioning section, hindrance of a visual field of a surgeon by the transitioning section. Further, by inserting only the wire through the transitioning section having the hollow pipe shape, it is possible to reduce backlash of connection of the members compared with when a plurality of members are connected and it is possible to appropriately transmit a precise movement of the handle section by the surgeon to the treatment instrument.

### BRIEF DESCRIPTION OF DRAWINGS

FIG. 1 is a perspective view illustrating a state in which a surgeon is grasping surgical equipment in an embodiment.

FIG. 2 is a front view of the surgical equipment.

FIG. 3 is a top view of the surgical equipment.

FIG. 4 is a perspective view illustrating a state in which the surgical equipment is viewed from rear of a handle section.

FIG. 5(A) is a main part sectional view illustrating a state in which a front end portion of a wire moves in a direction separating away from the handle section, whereby a treatment instrument (forceps) operates to be in a first posture (an open posture) and FIG. 5(B) is a main part sectional view illustrating a state in which the front end portion of the wire moves in a direction approaching the handle section, whereby the treatment instrument (the forceps) operates to be in a second posture (a closed posture).

FIG. 6 is a perspective view illustrating a state before the treatment instrument (the forceps) is attached to a front end portion of a pipe section

FIG. 7(A) is a front view illustrating main parts of a transitioning section and the handle section with a part of grip members of a manipulator section broken, FIG. 7(B) is a sectional view illustrating the main parts of the transitioning section and the handle section and illustrating a state in which the grip members of the manipulator section are separated away from a second axis parallel to the longitudinal direction of the handle section, and FIG. 7(C) is a sectional view illustrating the main parts of the transitioning section and the handle section and illustrating a state in which the grip members of the manipulator section approach the second axis parallel to the longitudinal direction of the handle section.

FIG. 8 is an explanatory diagram illustrating a procedure for assembling a wire, a ball member, and a slider in a transmission member.

### DESCRIPTION OF EMBODIMENT

A mode for carrying out the present invention is explained in detail below with reference to the drawings. An embodiment explained herein is illustrated to embody the technical idea of the present invention and does not limit the present invention. Accordingly, all of feasible other modes, examples, operation techniques, and the like conceivable by those skilled in the art and the like in a range not departing from the gist of the present invention are included in the scope and the gist of the present invention and included in the inventions described in the claims and the scope of equivalents of the inventions.

The drawings accompanying this specification are sometimes changed from actual objects and schematically expressed about scales, longitudinal and lateral dimension ratios, shapes, and the like as appropriate for convenience of illustration and easiness of understanding. However, the drawings are only examples and do not limit the interpretation of the present invention.

Note that, in this specification, ordinal numerals such as “first” and “second” are sometimes added. However, unless there is particular explanation concerning these ordinal numerals, the ordinal numerals are added in order to identify components for convenience of explanation and do not specify numbers or order.

In this specification, an end portion of a pipe section 10 on a side at which it is inserted first when being inserted into an organism is represented as a front end (a distal end) and an end portion on the other end side with respect to the front end portion is represented as a rear end (a proximal end). For elements other than the pipe section 10, front ends (distal ends) and rear ends (proximal ends) are defined in the same manner as a positional relation between the front end (the distal end) and the rear end (the proximal end) of the pipe section 10.

Concerning, an X axis, a Y axis, and a Z axis added to the figures, the X axis indicates a direction parallel to a longitudinal direction of a handle section 20, the Z axis indicates a direction parallel to a height direction of surgical equipment 1, and the Y axis indicates a direction orthogonal to the X axis and the Z axis.

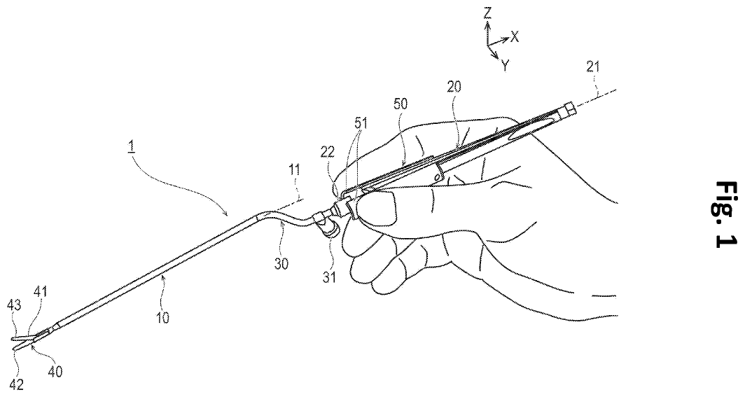

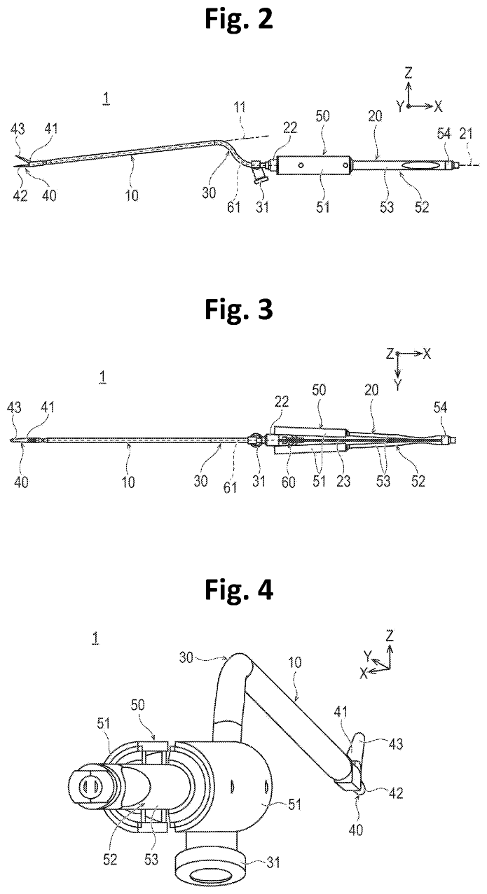

FIG. 1 is a perspective view illustrating a state in which a surgeon is grasping the surgical equipment 1 in the embodiment. FIG. 2 and FIG. 3 are a front view and a top view of the surgical equipment 1. FIG. 4 is a perspective view illustrating a state in which the surgical equipment 1 is viewed from rear of the handle section 20.

As illustrated in FIG. 1, FIG. 2, FIG. 3, and FIG. 4, generally speaking, the surgical equipment 1 in the embodiment includes the pipe section 10, the handle section 20, a transitioning section 30, a treatment instrument 40, a manipulator section 50, and a transmission member 60. The pipe section 10 is inserted into an organism. The handle section 20 extends in a direction nonparallel to a longitudinal direction of the pipe section 10. The transitioning section 30 couples a rear end portion of the pipe section 10 and a front end portion of the handle section 20. The treatment instrument 40 is disposed at a front end portion of the pipe section 10. The manipulator section 50 is disposed in the handle section 20 and manipulated when causing the treatment instrument 40 to operate. The transmission member 60 connects the treatment instrument 40 and the manipulator section 50 and transmits a motion of the manipulator section 50 to the treatment instrument 40. Here, the transmission member 60 includes a wire 61. The transitioning section 30 has a hollow pipe shape through which the wire 61 can be inserted. Only the wire 61 is inserted through the transitioning section 30. Detailed explanation is made below.

As illustrated in FIG. 2, in the front view of the surgical equipment 1, a first axis 11 parallel to the longitudinal direction of the pipe section 10 and a second axis 21 parallel to the longitudinal direction of the handle section 20 are shifted and are nonparallel. As illustrated in FIG. 3, in the top view of the surgical equipment 1, the first axis 11 of the pipe section 10 and the second axis 21 of the handle section 20 are parallel. The surgical equipment 1 has an exterior shape formed by shifting the first axis 11 of the pipe section 10 and the second axis 21 of the handle section 20 and coupling the pipe section 10 and the handle section 20 via the transitioning section 30. The transitioning section 30 has a gently curving pipe shape in the front view of the surgical equipment 1 (FIG. 2) and has a linear pipe shape in the top view of the surgical equipment 1 (FIG. 3). In the embodiment, the pipe section 10 and the transitioning section 30 is integrally formed by bending one pipe material. An outer diameter dimension of the pipe section 10 and the transitioning section 30 is not particularly limited but is, for example, approximately 3 mm in diameter. Note that the pipe section 10 and the transitioning section 30 separately formed may be joined by welding. A connecting section 31 for connecting a not-illustrated cleaning device is attached to the transitioning section 30. Cleaning liquid supplied from the cleaning device is injected into the transitioning section 30 from the connecting section 31.

The treatment instrument 40 is not particularly limited but is forceps 41 in the embodiment. A size of the forceps 41 is slightly smaller than the outer diameter dimension of the pipe section 10. For example, height and width dimensions of the forceps 41 are approximately 2 mm. The forceps 41 are attached to the front end portion of the pipe section 10 in a state in which the forceps 41 are not rotational around the first axis 11 of the pipe section 10.

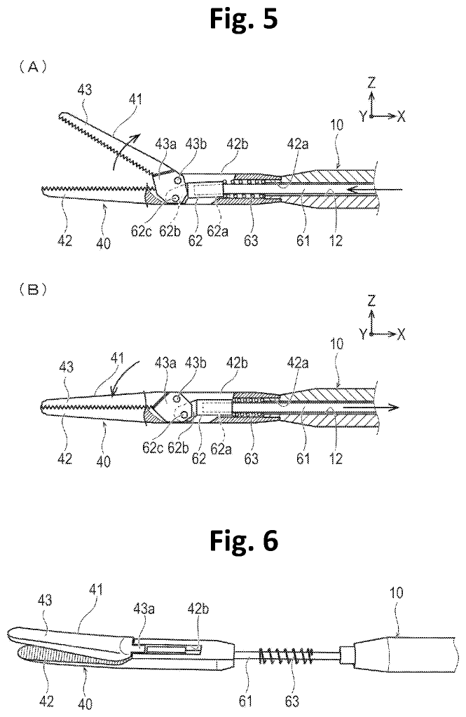

FIG. 5(A) is a main part sectional view illustrating a state in which a front end portion of the wire 61 moves in a direction separating away from the handle section 20, whereby the treatment instrument 40 (the forceps 41) operates to be in a first posture (for example, an open posture). FIG. 5(B) is a main part sectional view illustrating a state in which the front end portion of the wire 61 moves in a direction approaching the handle section 20, whereby the treatment instrument 40 (the forceps 41) operates to be in a second posture (for example, a closed state). FIG. 6 is a perspective view illustrating a state before the treatment instrument 40 (the forceps 41) is attached to the front end portion of the pipe section 10.

As illustrated in FIG. 5(A) and FIG. 5(B), the forceps 41 include a lower jaw 42 (equivalent to a fixed-side member) fixed to the pipe section 10 and an upper jaw 43 (equivalent to a moving-side member) to which the front end portion of the wire 61 is connected, the upper jaw 43 being capable of approaching and separating away from the lower jaw 42. A rear end side of the upper jaw 43 includes a plate-shaped base 43a. The base 43a of the upper jaw 43 is attached to the lower jaw 42 rotatably via a first pin 43b. A rear end side of the lower jaw 42 includes a through-hole 42a through which the wire 61 is inserted. The front end portion of the pipe section 10 is inserted into the through-hole 42a of the lower jaw 42. A rear end portion of the lower jaw 42 and the front end portion of the pipe section 10 are fixed by welding. The forceps 41 are attached to the front end portion of the pipe section 10 by weld-joining in a state in which the forceps 41 are not rotational around the first axis 11 of the pipe section 10. As illustrated in FIG. 6 as well, the base 43a of the upper jaw 43 is inserted into a groove section 42b formed in the lower jaw 42.

As illustrated in FIG. 5(A) and FIG. 5(B), the front end portion of the wire 61 is coupled to a front end metal fitting 62. A rear end side of the front end metal fitting 62 includes an attachment hole 62a. The front end portion of the wire 61 is inserted into the attachment hole 62a of the front end metal fitting 62 and fixed. A front end side of the front end metal fitting 62 includes a plate-shaped attachment plate 62b. The attachment plate 62b of the front end metal fitting 62 is fit in an attachment groove (not illustrated) formed in the base 43a of the upper jaw 43. The attachment plate 62b of the front end metal fitting 62 is attached to the base 43a of the upper jaw 43 rotatably via a second pin 62c. The wire 61 is inserted through a passage 12 of the pipe section 10 through the through-hole 42a of the lower jaw 42.

As illustrated in FIG. 5(A) and FIG. 5(B), a spring 63 (equivalent to a second spring member 63) is disposed between a rear end portion of the front end metal fitting 62 and the front end portion of the pipe section 10. As illustrated in FIG. 6, the spring 63 is put on the wire 61. The spring 63 applies, to the wire 61, an elastic force for moving the front end portion of the wire 61 connected to the forceps 41 in a left direction in the figure (the direction separating away from the handle section 20).

The first pin 43b and the second pin 62c are disposed to shift in the Z direction. As illustrated in FIG. 5(A), when the front end portion of the wire 61 moves in the left direction in the figure (the direction separating away from the handle section 20) with the elastic force of the spring 63, the upper jaw 43 rotates in a clockwise direction centering on the first pin 43b and moves to separate away from the lower jaw 42. Consequently, the forceps 41 operate to be in the first posture (the open posture). As illustrated in FIG. 5(B), when the front end portion of the wire 61 moves in a right direction in the figure (the direction approaching the handle section 20) resisting the elastic force of the spring 63, the upper jaw 43 rotates in a counterclockwise direction centering on the first pin 43b and moves to approach the lower jaw 42. Consequently, the forceps 41 operate to be in the second posture (the closed posture).

The forceps 41 and the pipe section 10 are assembled as explained below. The attachment plate 62b of the front end metal fitting 62 is fit in the attachment groove (not illustrated) of the upper jaw 43. The front end portion of the wire 61 is fixed to the front end metal fitting 62. The attachment plate 62b of the front end metal fitting 62 is attached to the base 43a of the upper jaw 43 rotatably via the second pin 62c. A rear end portion of the wire 61 is inserted through the groove section 42b and the through-hole 42a of the lower jaw 42. The base 43a of the upper jaw 43 is inserted into the groove section 42b of the lower jaw 42 (FIG. 6). The base 43a of the upper jaw 43 is attached to the lower jaw 42 rotatably via the first pin 43b. The spring 63 is put on the wire 61 and the rear end portion of the wire 61 is inserted through the passage 12 of the pipe section 10. The front end portion of the pipe section 10 is inserted into the through-hole 42a of the lower jaw 42. The rear end portion of the lower jaw 42 and the front end portion of the pipe section 10 are weld-joined. Consequently, the assembly of the forceps 41 and the pipe section 10 is completed.

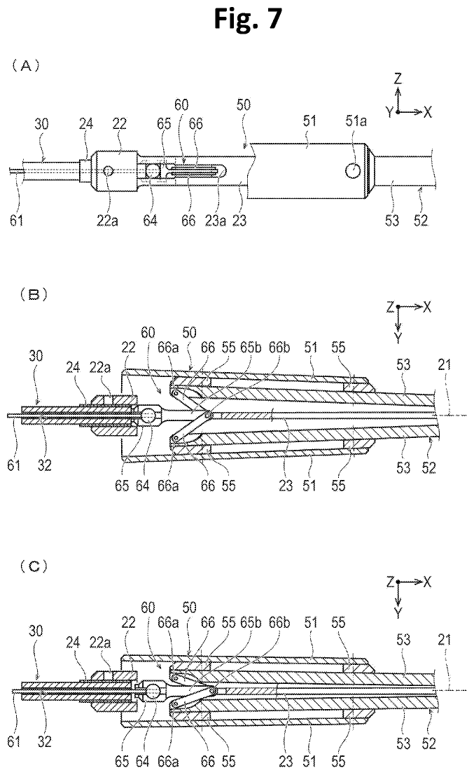

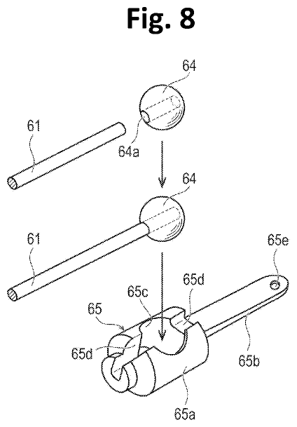

FIG. 7(A) is a front view illustrating main parts of the transitioning section 30 and the handle section 20 with a part of grip members 51 of the manipulator section 50 broken. FIG. 7(B) is a sectional view illustrating a state in which the grip members 51 of the manipulator section 50 are separated away from the second axis 21 of the handle section 20. FIG. 7(C) is a sectional view illustrating a state in which the grip members 51 of the manipulator section 50 approach the second axis 21 of the handle section 20. FIG. 8 is an explanatory diagram illustrating assembly order of the wire 61, a ball member 64, and a slider 65 in the transmission member 60.

As illustrated in FIG. 7(A), FIG. 7(B), and FIG. 7(C), the transitioning section 30 is formed from a pipe material in which a center hole 32 is formed. The wire 61 is inserted through the center hole 32.

The handle section 20 includes a tubular member 22 attached to a rear end portion of the transitioning section 30 and a center guide 23 extending from the tubular member 22. The tubular member 22 is screw-fastened to the rear end portion of the transitioning section 30 via a collar 24. A reference sign 22a indicates a through-hole formed in the tubular member 22. A screw (not illustrated) for fastening the tubular member 22 to the transitioning section 30 is inserted through the through-hole 22a. A long hole 23a is formed at a front end portion of the center guide 23.

The manipulator section 50 includes the grip members 51 capable of approaching and separating away from the second axis 21 of the handle section 20 and a first spring member 52 that applies, to the grip members 51, an elastic force for moving the grip members 51 in a direction separating away from the second axis 21. The first spring member 52 in the embodiment is formed from a V spring 53 having a V shape in the top view of the surgical equipment 1 (see FIG. 3 as well). A rear end portion of the V spring 53 holds a rear end portion of the center guide 23 of the handle section 20 and is fixed to the center guide 23 by a nut 54 (FIG. 3). A pair of front end portions of the V spring 53 is disposed with a gap between the pair of front end portions and the center guide 23 (FIG. 7(B) and FIG. 7(C)). The grip members 51 are screw-fastened to each of the pair of front end portions of the V spring 53 via spacers 55. Each of a pair of the grip members 51 has an arcuate shape in a cross section orthogonal to the longitudinal direction. A reference sign 51a in FIG. 7(A) indicates a through-hole formed in the grip members 51. A screw for fastening the grip members 51 to the V spring 53 is inserted into the through-hole 51a. As illustrated in FIG. 7(B), the grip members 51 move to positions separated away from the second axis 21 with an elastic force of the V spring 53. When the surgeon grasps the grip members 51, as illustrated in FIG. 7(C), the grip members 51 move to positions close to the second axis 21 resisting the elastic force of the V spring 53.

The transmission member 60 includes the wire 61, the ball member 64, the slider 65, a pair of link members 66, and a second spring member 63 (see FIG. 5(A) and FIG. 5(B)). The ball member 64 is coupled to the rear end portion of the wire 61. The slider 65 holds the ball member 64 and is slidable along the second axis 21. The pair of link members 66 connects the slider 65 to each of the pair of grip members 51 of the manipulator section 50. The second spring member is configured from the spring 63 as explained above and applies, to the wire 61, the elastic force for moving the front end portion of the wire 61 connected to the forceps 41 in the direction separating away from the handle section 20.

As illustrated in FIG. 8, a through-hole 64a is formed in the ball member 64. The rear end portion of the wire 61 is inserted through the through-hole 64a. The ball member 64 and the wire 61 are fixed by welding. At this time, a length of the wire 61 can be adjusted. The slider 65 includes a main body section 65a having a substantially cylindrical shape and a plate section 65b extending from a rear end portion of the main body section 65a. A recess 65c for housing the ball member 64 is formed in the main body section 65a of the slider 65. A groove section 65d is formed on an outer circumferential surface of the main body section 65a of the slider 65. The slider 65 is slidable along the second axis 21 with the groove section 65d fit in an inner peripheral edge of the long hole 23a of the center guide 23 (FIG. 7(A)). The ball member 64 is only fit in the recess 65c of the slider 65. That is, the ball member 64 is slidably housed in the recess 65c of the slider 65. A coupling hole 65e for coupling one ends of the link members 66 is formed at a rear end portion of the plate section 65b of the slider 65.

One of the link members 66 couples the front end portion of the V spring 53 illustrated on an upper side in FIG. 7(B) and FIG. 7(C) and the plate section 65b of the slider 65. The other of the link members 66 couples the front end portion of the V spring 53 illustrated on a lower side in FIG. 7(B) and FIG. 7(C) and the plate section 65b of the slider 65. Front end portions of the respective link members 66 are coupled to the respective front end portions of the V spring 53 rotatably via third pins 66a. Rear end portions of the respective link members 66 are coupled to the coupling hole 65e of the plate section 65b rotatably via a fourth pin 66b. The grip members 51 are attached to the respective front end portions of the V spring 53. Therefore, the pair of link members 66 comes into a state in which the pair of link members 66 connects the slider 65 and each of the pair of grip members 51. Each of the pair of link members is always inclined with respect to the second axis 21 of the handle section 20.

A material forming the members explained above configuring the surgical equipment 1 is not particularly limited. However, members requiring rigidity, for example, the pipe materials configuring the pipe section 10 and the transitioning section 30, the treatment instrument 40, and the wire 61 are formed from a metal material, for example, stainless steel (SUS 304). Members requiring fit feeling, for example, the grip members 51 may be formed from a resin material, rubber, or the like.

Next, an operation of the surgical equipment 1 is explained.

In neuroendoscopic surgery, an endoscope made of metal and the surgical equipment 1 are inserted into a lumen of a transparent sheath detained transcranially. As illustrated in FIG. 1, the surgeon grasps the surgical equipment 1 by holding the pair of grip members 51 with a thumb and an index finger.

Until the surgeon applies a predetermined force to move the grip members 51, as illustrated in FIG. 7(B), the grip members 51 move to positions separated away from the second axis 21 with the elastic force of the V spring 53. In this state, as illustrated in FIG. 5(A), the front end portion of the wire 61 is moved in the left direction in the figure (the direction separating away from the handle section 20) by the elastic force of the spring 63. The upper jaw 43 rotates in the clockwise direction centering on the first pin 43b and moves to separate away from the lower jaw 42. Consequently, the forceps 41 operate to be in the first posture (the open posture). As illustrated in FIG. 7(B), the slider 65 connected to the grip members 51 via the link members 66 slides toward the rear end portion of the transitioning section 30.

When the surgeon applies the predetermined force to move the grip members 51, as illustrated in FIG. 7(C), the grip members 51 move to positions close to the second axis 21 resisting the elastic force of the V spring 53. In this state, as illustrated in FIG. 7(C), the slider 65 connected to the grip members 51 via the link members 66 slides toward the rear end portion of the handle section 20 along the second axis 21. As illustrated in FIG. 5(B), the front end portion of the wire 61 is moved in the right direction in the figure (the direction approaching the handle section 20) resisting the elastic force of the spring 63. The upper jaw 43 rotates in the counterclockwise direction centering on the first pin 43b and moves to approach the lower jaw 42. Consequently, the forceps 41 operate to be in the second posture (the closed posture) different from the first posture (the open posture).

As explained above, the surgical equipment 1 in the embodiment has the exterior shape formed by shifting the axis (the first axis 11) of the pipe section 10 and the axis (the second axis 21) of the handle section 20 and coupling the pipe section 10 and the handle section 20 via the transitioning section 30. The transmission member 60 includes the wire 61. The transitioning section 30 has the hollow pipe shape through which the wire 61 can be inserted. Only the wire 61 is inserted through the transitioning section 30.

With the surgical equipment 1 configured as explained above, since the transitioning section 30 has a pipe shape, the transitioning section 30 can be reduced in size. It is possible to provide the surgical equipment 1 that reduces, through the reduction in the size of the transitioning section 30, hindrance of a visual field of the surgeon by the transitioning section 30. Further, by inserting only the wire 61 through the transitioning section 30 having the hollow pipe shape, it is possible to reduce backlash of connection of the members compared with when a plurality of members are connected and it is possible to appropriately transmit a precise movement of the handle section 20 by the surgeon to the treatment instrument 40. It is possible to sufficiently secure a surgical visual field when inserting the surgical equipment 1 into the sheath. Since the transitioning section 30 can be reduced in size, it is possible to move the center of gravity of the surgical equipment 1 to the near side. The surgeon feels more stability when holding the surgical equipment 1. Further, since the transitioning section 30 has a structure through which only the wire 61 is inserted, it is possible to easily cope with shape changes for a shift amount, an angle, a length, and the like between the first axis 11 of the pipe section 10 and the second axis 21 of the handle section 20. As a result, it possible to contribute to a flexible design when variations of the treatment instrument 40 are expanded.

The treatment instrument 40 is attached to the front end portion of the pipe section 10 in a state in which the treatment instrument 40 is not rotational around the first axis 11 parallel to the longitudinal direction of the pipe section 10.

With such a configuration, a mechanism for rotating the treatment instrument 40 around the first axis 11 is unnecessary. The number of components can be reduced. As a result, it is possible to achieve a further reduction in the size of the transitioning section 30. It is possible to further reduce hindrance of the visual field of the surgeon by the transitioning section 30. Since the number of components decreases, it is possible to reduce a backlash amount when causing the treatment instrument 40 to operate. Since the number of components decreases, it is possible to provide the surgical equipment 1 advantageous in terms of cost and it is possible to reduce occurrence of failures.

The treatment instrument 40 includes the lower jaw 42 (the fixed-side member) fixed to the pipe section 10 and the upper jaw 43 (the moving-side member) to which the front end portion of the wire 61 is connected, the upper jaw 43 being capable of approaching and separating away from the fixed-side member.

With such a configuration, only the upper jaw 43 has to be caused to operate by traction of the wire 61. It is possible to simplify a configuration for driving the treatment instrument 40 and it is possible to reduce the number of components. As a result, it is possible to achieve a further reduction in the size of the transitioning section 30. It is possible to further reduce hindrance of the visual field of the surgeon by the transitioning section 30. Since the number of components decreases, it is possible to reduce a backlash amount when causing the treatment instrument 40 to operate. Since the number of components decreases, it is possible to provide the surgical equipment 1 advantageous in terms of cost. It is possible to reduce occurrence of failures.

The manipulator section 50 includes the grip members 51 and the V spring 53 (the first spring member 52). The transmission member 60 includes the ball member 64, the slider 65, the link members 66, and the spring 63 (the second spring member). In a state in which the grip members 51 are separated away from the second axis 21 by the elastic force of the V spring 53, the front end portion of the wire 61 is moved in the direction separating away from the handle section 20 by the elastic force of the spring 63 and the treatment instrument 40 operates to be in the first posture (for example, the open posture). In a state in which the grip members 51 approach the second axis 21 resisting the elastic force of the V spring 53, the slider 65 connected to the grip members 51 via the link members 66 moves toward the rear end portion of the handle section 20 along the second axis 21, the front end portion of the wire 61 is moved in the direction approaching the handle section 20 resisting the elastic force of the spring 63, and the treatment instrument 40 operates to be in the second posture (for example, the closed posture) different from the first posture.

With such a configuration, even if a wire power transmission system is adopted, it is possible to surely cause the treatment instrument 40 to operate to be in the first posture and the second posture. Since the wire 61 is always pulled to the front end side by the elastic force of the spring 63, a slack of the wire 61 causing backlash of the treatment instrument 40 does not occur. Consequently, when the manipulator section 50 is manipulated, it is possible to cause the treatment instrument 40 to operate without delay.

The ball member 64 of the transmission member 60 is coupled to the rear end portion of the wire 61 by welding.

With such a configuration, when the surgical equipment 1 is assembled, it is possible to adjust the length of the wire 61. Even if the wire power transmission system is adopted, since the length of the wire 61 can be adjusted, a situation in which the wire 61 slacks when the surgical equipment 1 is assembled does not occur. As a result, it is possible to more surely operate the treatment instrument 40 to be in the first posture and the second posture.

The ball member 64 of the transmission member 60 is slidably housed in the recess 65c formed in the slider 65.

With such a configuration, since the slider 65 functions as a structure for receiving the ball member 64, it is possible to absorb a twist that occurs when assembling the surgical equipment 1 and a twist of the wire 61 that occurs when causing the wire 61 to operate. As a result, it is possible to more surely operate the treatment instrument 40 to be in the first posture and the second posture. Further, since the slider 65 functions as the structure for receiving the ball member 64, it is easy to assemble the surgical equipment 1.

The treatment instrument 40 is the forceps 41.

With such a configuration, it is possible to perform predetermined treatment for a diseased part with the forceps 41 while reducing hindrance of the visual field of the surgeon by the transitioning section 30.

The surgical equipment 1 according to the present invention is explained above through the embodiment. However, the present invention is not limited to only the explained configurations and can be changed as appropriate based on the description of the claims.

For example, the exterior shape formed by shifting the first axis 11 of the pipe section 10 and the second axis 21 of the handle section 20 is not limited to the illustrated form and can be set to a different exterior shape as desired. The shape of the transitioning section 30 is also not limited to the illustrated form. The transitioning section 30 can have a pipe shape that not only curves in the front view of the surgical equipment 1 but also curves in the top view of the surgical equipment 1.

The treatment instrument 40 is not limited to the forceps 41. For example, the treatment instrument 40 may be biopsy forceps, tweezers, scissors, a needle holder, or the like having a function of cutting cells.

The V spring 53 is illustrated as the first spring member 52. However, the first spring member 52 can be configured from a spring like the second spring member.

### REFERENCE SIGNS LIST

1: surgical equipment
10: pipe section
11: first axis
20: handle section
21: second axis
30: transitioning section
32: center hole
40: treatment instrument
41: forceps
42: lower jaw (fixed-side member)
43: upper jaw (moving-side member)
50: manipulator section
51: grip member
52: first spring member
53: V spring (first spring member)
60: transmission member
61: wire
62: front end metal fitting
63: spring (second spring member)
64: ball member
65: slider
65c: recess
66: link member

1. Surgical equipment comprising:
a pipe section to be inserted into an organism;
a handle section extending in a direction nonparallel to a longitudinal direction of the pipe section;
a transitioning section that couples a rear end portion of the pipe section and a front end portion of the handle section;
a treatment instrument disposed at a front end portion of the pipe section;
a manipulator section disposed in the handle section and manipulated when causing the treatment instrument to operate; and
a transmission member that connects the treatment instrument and the manipulator section and transmits a motion of the manipulator section to the treatment instrument, wherein
the transmission member includes a wire, and
the transitioning section has a hollow pipe shape through which the wire can be inserted, and only the wire is inserted through the transitioning section.

2. The surgical equipment according to claim 1, wherein the treatment instrument includes: a fixed-side member fixed to the pipe section; and a moving-side member to which a front end portion of the wire is connected, the moving-side member being capable of approaching and separating away from the fixed-side member.

3. The surgical equipment according to claim 1, wherein
the manipulator section includes: a grip member capable of approaching and separating away from a second axis parallel to a longitudinal direction of the handle section; and a first spring member that applies, to the grip member, an elastic force for moving the grip member in a direction separating away from the second axis,
the transmission member includes: a ball member coupled to a rear end portion of the wire; a slider that holds the ball member and is slidable along the second axis; a link member that connects the slider to the grip member of the manipulator section; and a second spring member that applies, to the wire, an elastic force for moving the front end portion of the wire connected to the treatment instrument in a direction separating away from the handle section,
in a state in which the grip member is separated away from the second axis by the elastic force of the first spring member, the front end portion of the wire is moved in a direction separating away from the handle section by the elastic force of the second spring member and the treatment instrument operates to be in a first posture, and
in a state in which the grip member approaches the second axis resisting the elastic force of the first spring member, the slider connected to the grip member via the link member moves toward a rear end portion of the handle section along the second axis, the front end portion of the wire is moved in a direction approaching the handle section resisting the elastic force of the second spring member, and the treatment instrument operates to be in a second posture different from the first posture.

4. The surgical equipment according to claim 3, wherein the ball member of the transmission member is coupled to the rear end portion of the wire by welding.

5. The surgical equipment according to claim 3, wherein the ball member of the transmission member is slidably housed in a recess formed in the slider.

6. The surgical equipment according to claim 1, wherein the treatment instrument is forceps.
